(19) United States
(12) Patent Application Publication
(10) Pub. No.: US 2021/0251876 A1
(43) Pub. Date: Aug. 19, 2021

(54) UV PROTECTION COSMETIC COMPOSITION

(71) Applicant: KOLMAR KOREA CO., LTD., Sejong (KR)

(72) Inventors: Jin Young KIM, Seoul (KR); Su Bin CHOI, Seoul (KR); Kyoung Jin KIM, Seoul (KR); Sung Eun HONG, Seoul (KR); Sung Bong KYE, Yongin-si, Gyeonggi-do (KR)

(21) Appl. No.: 16/769,456

(22) PCT Filed: Oct. 29, 2019
(86) PCT No.: PCT/KR2019/014397

§ 371 Date: Jun. 3, 2020

(30) Foreign Application Priority Data
Apr. 3, 2019 (KR) 10-2019-0039162

(51) Int. Cl. A61K 8/895; A61K 8/81; A61K 8/04; A61K 8/34; A61K 8/67; A61K 8/25; A61K 8/58; A61K 8/37; A61K 8/40; A61K 8/891; A61K 8/73; A61K 8/41; A61Q 17/04

(52) U.S. Cl. CPC A61K 8/895; A61K 2800/48; A61K 8/042; A61K 8/34; A61K 8/678; A61K 8/25; A61K 8/585; A61K 8/37; A61K 8/40; A61K 8/891; A61K 8/731; A61K 8/415; A61Q 17/04; A61K 2800/412; A61K 8/8176

(57) ABSTRACT

A UV protection cosmetic composition is provided. The composition may include an organic UV protection agent, a thickening agent, a silicone elastomer, ethanol and oil.

### BACKGROUND

### 1. Field of the Invention

The present invention relates to a UV protection cosmetic composition, and specifically, to a gel-type UV protection cosmetic composition having excellent usability and an excellent UV protection function without white cast.

### 2. Discussion of Related Art

Recently, due to the destruction of the ozone layer caused by environmental pollution, the amount of UV light reaching the surface of the Earth has increased, and thus the general public's interest in the effects of UV light on the human body has increased considerably. Accordingly, various UV protection products have been released. In particular, consumers' awareness of the need for UV protection in everyday life is expanding, not only during summer or sports and leisure activities. Due to climate changes, the climate of Korea is changing to a subtropical climate, increasing the demand of consumers for UV protection products with fresh and light feeling upon use.

Generally, UV protection products may be prepared in a variety of formulations, such as cream, lotion, cake, stick and powder types. Among them, oil-type UV protection agents and sun-stick products formed by hardening emollients in solid form have received excellent reviews from consumers because of transparent skin expression, good portability and re-application. However, in the case of such a transparent sun-stick product, it is oily or sticky after use due to containing a large amount of oil ingredients, and it is difficult to apply to curved skin. Furthermore, the UV protection product prepared in a stick formulation is unable to provide a cool feeling when applied to skin in summer, due to containing no water or a very small amount of water.

The UV protection product prepared in an oil formulation with low viscosity has poor usability due to severe oiliness or stickiness, and when the oil formulation is applied to a cosmetic container, leakage occurs between a container gap due to low surface tension, causing problems of storage and portability.

Further, since the existing UV protection agent contains a large amount of emulsifier, there is a problem in that the UV protection agent is re-emulsified on contact with water, causing the occurrence of white cast forming a white milky layer.

Therefore, there is a need to develop a UV protection cosmetic composition to solve the existing problems.

### SUMMARY OF THE INVENTION

To address the above-discussed deficiencies, an object of the present invention is to provide a gel-type UV protection cosmetic composition having excellent usability and an excellent UV protection function without white cast.

The technical objects of the present invention are not limited to the above-mentioned objects, and other technical objects may be clearly understood by those skilled in the art from the disclosure below.

According to an embodiment of the present invention, a UV protection cosmetic composition including an organic UV protection agent, a thickening agent, a silicone elastomer, ethanol and oil may be provided.

Preferably, the silicone elastomer may be in powder form.

Further, preferably, the silicone elastomer may include one or more selected from the group consisting of dimethicone crosspolymer, dimethicone/vinyl dimethicone crosspolymer, dimethicone/vinyl dimethicone crosspolymer-silica, dimethicone/vinyl dimethicone crosspolymer-silica-dimethicone, vinyl dimethicone/methicone silsesquioxane crosspolymer and a mixture thereof.

Further, preferably, the thickening agent may include polyvinyl pyrrolidone.

Further, preferably, the thickening agent may further include one or more selected from the group consisting of hydroxypropyl cellulose, hydroxypropyl methylcellulose, hydroxypropyl methylcellulose stearoxy ether, acrylate/C10-30 alkyl acrylate crosspolymer, carbomer, hydroxyethyl acrylate/sodium acryloyldimethyl taurate copolymer, polyacrylate-13, polyvinyl alcohol, ammonium acryloyldimethyl taurate/VP copolymer, polyacrylate crosspolymer-6, polyacrylate crosspolymer-11, an acrylate copolymer and a mixture thereof.

Further, preferably, the organic UV protection agent may include one or more selected from the group consisting of ethylhexyl methoxycinnamate, ethylhexyl salicylate, homosalate, octocrylene, polysilicone-15, isoamyl p-methoxycinnamate, ethylhexyl triazone, butyl methoxydibenzoylmethane, bis-ethylhexyloxyphenol methoxyphenyl triazine, diethylamino hydroxybenzoyl hexyl benzoate, 4-methylbenzylidene camphor, benzophenone-3, phenylbenzimidazole sulfonic acid, drometrizole trisiloxane, terephthalylidene dicamphor sulfonic acid, disodium phenyl dibenzimidazole tetrasulfonate and a mixture thereof.

Further, preferably, the oil may include one or more selected from the group consisting of butyloctyl salicylate, dibutyl adipate, butylene glycol dicaprylate/dicaprate, propylene glycol dicaprylate/dicaprate, propylene glycol dicaprylate, dicaprylyl carbonate, propylheptyl caprylate, dicaprylyl ether, coco-caprylate/caprate, ethylhexyl palmitate, cyclopentasiloxane, dimethicone, disiloxane, trisiloxane, ethyl methicone, methyl trimethicone, phenyl trimethicone, caprylyl methicone, isododecane, isoeicosane, C15-19 alkanes and a mixture thereof.

Further, preferably, the composition may include, based on the total weight of the composition, 1 to 60 wt % of the organic UV protection agent, 0.05 to 20 wt % of the thickening agent, 0.05 to 30 wt % of the silicone elastomer in powder form, 1 to 80 wt % of the ethanol and 1 to 60 wt % of the oil.

Further, preferably, the composition may include, based on the total weight of the composition, 5 to 40 wt % of the organic UV protection agent, 0.1 to 8 wt % of the thickening agent, 0.1 to 10 wt % of the silicone elastomer in powder form, 4 to 60 wt % of the ethanol and 5 to 40 wt % of the oil.

Further, preferably, a particle diameter of the powder particle of the silicone elastomer may be in a range of 1 to 20 micrometers.

Further, preferably, the composition may be a gel formulation.

Further, preferably, the composition may have a viscosity of 24,000 cP or more.

Further, preferably, the composition may have a viscosity ranging from 30,000 to 100,000 cP.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a view showing a composition formulation of Example 2; and

FIG. 2 is a view showing a composition formulation of Example 8.

### DETAILED DESCRIPTION OF EXEMPLARY EMBODIMENTS

Unless defined otherwise, all technical and scientific terms used herein have the same meaning as commonly understood by one of ordinary skill in the art to which the invention pertains. Generally, the nomenclature used herein is well known and is commonly employed in the art. Further, in the description of embodiments of the present invention, a detailed description of known functions and configurations incorporated herein will be omitted to avoid making the subject matter of the present invention unclear. Further, although embodiments of the present invention are described below, the invention may be embodied in many alternate forms by those skilled in the art without departing from the spirit of the invention and should not be construed as limited to only the embodiments set forth herein.

A UV protection cosmetic composition according to the present invention may include an organic UV protection agent, a thickening agent, a silicone elastomer, ethanol and oil.

Hereinafter, each ingredient of the UV protection cosmetic composition of the present invention will be described in further detail.

The organic UV protection agent of the present invention is a composition having a UV protection effect, and an organic UV protection agent commonly used in the related field may be used. More specifically, the organic UV protection agent may include one or more selected from the group consisting of ethylhexyl methoxycinnamate, ethylhexyl salicylate, homosalate, octocrylene, polysilicone-15, isoamyl p-methoxycinnamate, ethylhexyl triazone, butyl methoxydibenzoylmethane, bis-ethylhexyloxyphenol methoxyphenyl triazine, diethylamino hydroxybenzoyl hexyl benzoate, 4-methylbenzylidene camphor, benzophenone-3, phenylbenzimidazole sulfonic acid, drometrizole trisiloxane, terephthalylidene dicamphor sulfonic acid, disodium phenyl dibenzimidazole tetrasulfonate and a mixture thereof.

In the present invention, the organic UV protection agent may be included in an amount of 1 to 60 wt %, preferably 5 to 40 wt %, more preferably 7 to 35 wt %, and most preferably 10 to 30 wt % based on the total weight of the composition. When the organic UV protection agent is included in an amount of less than 1 wt %, the UV protection effect is insignificant. When the organic UV protection agent composition is included in an amount of more than 60 wt %, the organic UV protection agent composition may be precipitated in the form of granules or cause stickiness, resulting in a decrease in usability.

In an embodiment, the organic UV protection agent may include, based on the total weight of the composition, 2.0 to 20.0 wt % of ethylhexyl methoxycinnamate, 2.0 to 15.0 wt % of ethylhexyl salicylate, 0.1 to 15.0 wt % of homosalate, 0.1 to 12.0 wt % of octocrylene, 0.1 to 10.0 wt % of polysilicone-15, 0.1 to 20.0 wt % of isoamyl p-methoxycinnamate, 0.1 to 10.0 wt % of ethylhexyl triazone, 0.1 to 10.0 wt % of butyl methoxydibenzoylmethane, 0.1 to 10.0 wt % of bis-ethylhexyloxyphenol methoxyphenyl triazine, 0.1 to 10.0 wt % of diethylamino hydroxybenzoyl hexyl benzoate, 0.1 to 4.0 wt % of 4-methylbenzylidene camphor, 0.1 to 5.0 wt % of benzophenone-3, 0.1 to 8.0 wt % of phenylbenzimidazole sulfonic acid, 0.1 to 15.0 wt % of drometrizole trisiloxane, 0.1 to 10.0 wt % of terephthalylidene dicamphor sulfonic acid and 0.1 to 10.0 wt % of disodium phenyl dibenzimidazole tetrasulfonate, but the present invention is not limited thereto.

The thickening agent of the present invention is for controlling the viscosity of the composition and may include polyvinyl pyrrolidone (PVP).

Polyvinyl pyrrolidone is a thickening agent having a high thickening effect and may be used to improve the viscosity of the composition of the present invention.

Further, the thickening agent of the present invention may further include one or more thickening agents in addition to polyvinyl pyrrolidone. Since long-term viscosity retention may be reduced when polyvinyl pyrrolidone is used alone, the viscosity retention of the composition may be improved by adding a separate thickening agent having high compatibility with an organic UV protection agent and an oil ingredient and high viscosity retention in an ethanol dispersion.

The thickening agent which is mixable with polyvinyl pyrrolidone may be one or more selected from the group consisting of hydroxypropyl cellulose, hydroxypropyl methylcellulose, hydroxypropyl methylcellulose stearoxy ether, acrylates/C10-30 alkyl acrylate crosspolymer, carbomer, hydroxyethyl acrylate/sodium acryloyldimethyl taurate copolymer, polyacrylate-13, polyvinyl alcohol, ammonium acryloyldimethyl taurate/VP copolymer, polyacrylate crosspolymer-6, polyacrylate crosspolymer-11, an acrylate copolymer and a mixture thereof.

In the present invention, the thickening agent may be included in an amount of 0.05 to 20 wt %, preferably 0.1 to 8 wt %, more preferably 1 to 6 wt %, and most preferably 1.5 to 4 wt % based on the total weight of the composition. When the thickening agent is included in an amount of less than 0.05 wt %, the viscosity of the cosmetic is not maintained above a certain value, and thus it is difficult to maintain the gel form. When the thickening agent is included in an amount of more than 20 wt %, it is difficult to disperse the thickening agent.

The silicone elastomer of the present invention is for improving the viscosity of the composition and may preferably be in powder form. Specifically, the silicone elastomer powder, which is crosslinked, swells while absorbing oil, improving the viscosity of the composition.

In an embodiment, the silicone elastomer powder acts with the thickening agent to form and maintain the viscosity of the composition above a certain level, thereby stably maintaining the composition in a gel type.

Further, the silicone elastomer powder has a unique silky and elastic feeling, which can maximize usability.

Conventionally, powders such as silica, methyl methacrylate crosspolymer, polymethyl silsesquioxane and the like have a spherical shape and have oil absorption capacity, and thus are largely used to improve usability. However, when a large amount of oil is absorbed, powders harden without swelling, or when powders are blended with an amount of oil exceeding oil absorption capacity, there is a problem in that the oil absorbed is re-released to deteriorate the stability of the formulation.

In order to solve the problem, in the present invention, the thickening agent is mixed with the silicone elastomer powder to maintain a gel phase, to improve the stability of the formulation, and to greatly improve usability.

The silicone elastomer powder of the present invention may include one or more selected from the group consisting of dimethicone crosspolymer, dimethicone/vinyl dimethicone crosspolymer, dimethicone/vinyl dimethicone crosspolymer-silica, dimethicone/vinyl dimethicone crosspolymer-silica-dimethicone, vinyl dimethicone/methicone silsesquioxane crosspolymer and a mixture thereof.

In the present invention, the silicone elastomer powder may be included in an amount of 0.05 to 30 wt %, preferably 0.1 to 10 wt %, more preferably 1 to 8 wt %, and most preferably 2 to 6 wt % based on the total weight of the composition. When the silicone elastomer powder is included in an amount of less than 0.05 wt %, it is required to use an excess amount of the thickening agent, and in this case, the feeling is sticky, and it is difficult to disperse the thickening agent. When the silicone elastomer powder is included in an amount of more than 30 wt %, the silicone elastomer powder absorbs an excess amount of oil to reduce the solubility of the UV protection agent in the solid state, resulting in instability of the formulation.

The particle diameter of the silicone elastomer powder particle may be in a range of 0.5 to 20 micrometers, and preferably 1 to 10 micrometers. The silicone elastomer powder may have excellent oil absorption capacity and formulation stability when the particle diameter is in the aforementioned particle diameter range, but the present invention is not limited to the aforementioned range.

The ethanol of the present invention is for dispersing the organic thickening agent and may provide a cool feeling upon application to the skin.

In the present invention, the ethanol may be included in an amount of 1 to 80 wt %, preferably 4 to 60 wt %, more preferably 8 to 50 wt %, and most preferably 12 to 45 wt % based on the total weight of the composition. When the ethanol is included in an amount of less than 1 wt %, it is difficult to disperse the thickening agent, and it is impossible to provide a cool feeling upon application to the skin. When the ethanol is included in an amount of more than 80 wt %, the composition hardly adheres evenly to the skin due to high volatility, and viscosity retention may be lowered.

The oil of the present invention is for increasing the solubility of the organic UV protection agent and can improve the usability and spreadability of the composition.

More specifically, the oil may include one or more selected from the group consisting of butyloctyl salicylate, dibutyl adipate, butylene glycol dicaprylate/dicaprate, propylene glycol dicaprylate/dicaprate, propylene glycol dicaprylate, dicaprylyl carbonate, propylheptyl caprylate, dicaprylyl ether, coco-caprylate/caprate, ethylhexyl palmitate, cyclopentasiloxane, dimethicone, disiloxane, trisiloxane, ethyl methicone, methyl trimethicone, phenyl trimethicone, caprylyl methicone, isododecane, isoeicosane, C15-19 alkane and a mixture thereof.

In the present invention, the oil may be included in an amount of 1 to 60 wt %, preferably 5 to 40 wt %, more preferably 10 to 30 wt %, and most preferably 15 to 25 wt % based on the total weight of the composition. When the oil is included in an amount of less than 1 wt % based on the total weight of the composition, the solubility of the organic UV protection agent is not enhanced, and it is difficult to realize a feeling of pliability upon use. When the oil is included in an amount of more than 60 wt %, an excessive oil content decreases usability and absorption power, and formulation stability may be reduced, such as oil separation.

The UV protection cosmetic composition of the present invention may further include one or more selected from functional additives having the effect of whitening, antioxidant, wrinkle improvement, moisturizing, etc., skin conditioning agents, preservatives, fragrances, lubricants, antioxidants, discoloration inhibitors, stabilizers and other additives commonly used in the related field.

The form of the functional additive is not limited and may be implemented in various types such as a powder type, a liquid type, a capsule type, etc.

For example, wrinkle improvement functional additives may include hydrolyzed collagen, adenosine, etc.

Moisturizing functional additives may include propylene glycol, butylene glycol, dipropylene glycol, pentylene glycol, glycerin, etc.

Whitening functional additives may include oil-soluble licorice (glycyrrihiza) extract, bisabolol, niacinamide, etc.

Antioxidant functional additives and skin conditioning agents may include panthenol, tocopheryl acetate, hydroxyacetophenone, etc.

Preservatives may include phenoxy ethanol, ethylhexyl glycerin, etc.

Stabilizers may include PEG-10 dimethicone, lauryl PEG-9 polydimethylsiloxyethyl dimethicone, and lauryl PEG-10 tris(trimethylsiloxy) silylethyl dimethicone, polyglyceryl-3 polydimethyl siloxyethyl dimethicone, etc.

However, the present invention is not limited thereto, and various skin functional additives commonly used in the related field and ingredients included in general cosmetic compositions may be further included.

The UV protection cosmetic composition according to the present invention may include, based on the total weight of the composition, 1 to 60 wt % of the organic UV protection agent, 0.05 to 20 wt % of the thickening agent, 0.05 to 30 wt % of the silicone elastomer powder, 1 to 80 wt % of the ethanol and 1 to 60 wt % of the oil.

More specifically, the UV protection cosmetic composition according to the present invention may include, based on the total weight of the composition, 5 to 40 wt % of the organic UV protection agent, 0.1 to 8 wt % of the thickening agent, 0.1 to 10 wt % of the silicone elastomer powder, 4 to 60 wt % of the ethanol and 5 to 40 wt % of the oil.

When each ingredient of the UV protection cosmetic composition according to the present invention is included in an amount in the aforementioned range, the dissolution stability of the UV protection agent is excellent, water resistance is maintained, and a cool and refreshing feeling upon use can be provided.

The UV protection cosmetic composition according to the present invention may be a gel formulation. Specifically, the composition may be a gel formulation having a viscosity of 24,000 cP or more, preferably a viscosity in a range of 30,000 cP to 100,000 cP, and more preferably a viscosity in a range of 30,000 cP to 80,000 cP.

In the present invention, a gel type formulation can be formed and maintained due to the mixed use of the thickening agent and the silicone elastomer in powder form. Specifically, a certain level of viscosity is formed due to the thickening agent, and the silicone elastomer of the powder formulation can swell while absorbing oil to maintain a high viscosity.

Specifically, in the present invention, polyvinyl pyrrolidone and one or more thickening agents are mixed to form a stable viscosity for a long time, the swelling action of the silicone elastomer and oil enables the maintenance of complex viscosity, and thus a stable and high viscosity can be maintained for a long time.

As described above, since the composition of the present invention can maintain a viscosity above a certain value, there is no restriction on the storage container, and thus portability is excellent, re-application is easy, skin adhesion and persistence effects are high, and an excellent water resistance effect can be provided.

Hereinafter, examples for explaining the present invention in more detail are shown as follows, but the present invention is not limited thereto.

### Examples 1 to 6: Preparation of Composition not Containing Silicone Elastomer

A UV protection cosmetic composition was prepared using ingredients listed in the following Table 1.

Specifically, the organic UV protection agent was dissolved by heating at 80° C. and cooled to 25° C. to prepare phase A, and then oil was added to phase A to prepare phase B. Ethanol and a thickening agent were uniformly dispersed at room temperature using an AGI mixer to prepare phase C. After adding phase B to phase C, dispersion was performed at 4000 rpm for 5 minutes using a HOMO mixer.

Here, polyvinyl pyrrolidone was essentially included as a thickening agent, and was further mixed with an organic thickening agent, which is one of hydroxypropyl cellulose, hydroxypropyl methyl cellulose, hydroxypropyl methyl cellulose stearoxy ether, polyacrylate crosspolymer-6 and an acrylate copolymer, to prepare Examples 1 to 6.

TABLE 1

| Classification | Ingredient (wt %) | Example 1 | Example 2 | Example 3 | Example 4 | Example 5 | Example 6 |
|---|---|---|---|---|---|---|---|
| Organic UV | Ethylhexyl | 7.0 | 7.0 | 7.0 | 7.0 | 7.0 | 7.0 |
| protection | methoxycinnamate | | | | | | |
| agent | Ethylhexyl salicylate | 3.0 | 3.0 | 3.0 | 3.0 | 3.0 | 3.0 |
| | Homosalate | 4.0 | 4.0 | 4.0 | 4.0 | 4.0 | 4.0 |
| | Octocrylene | 3.0 | 3.0 | 3.0 | 3.0 | 3.0 | 3.0 |
| | Diethylamino | 1.0 | 1.0 | 1.0 | 1.0 | 1.0 | 1.0 |
| | hydroxybenzoyl hexyl | | | | | | |
| | benzoate | | | | | | |
| | Butyl | 2.0 | 2.0 | 2.0 | 2.0 | 2.0 | 2.0 |
| | methoxydibenzoylmethane | | | | | | |
| Oil | Butyl octyl salicylate | 10.0 | 10.0 | 10.0 | 10.0 | 10.0 | 10.0 |
| | Dicaprylyl carbonate | 6.5 | 6.0 | 6.0 | 6.0 | 6.0 | 6.0 |
| | Cyclopentasiloxane | 14.0 | 14.0 | 14.0 | 14.0 | 14.0 | 14.0 |
| | Tocopheryl acetate | 1.0 | 1.0 | 1.0 | 1.0 | 1.0 | 1.0 |
| Ethanol | Ethanol | 45.0 | 45.0 | 45.0 | 45.0 | 45.0 | 45.0 |
| Thickening | Polyvinyl pyrrolidone | 3.5 | 3.5 | 3.5 | 3.5 | 3.5 | 3.5 |
| agent | Hydroxypropyl cellulose | — | 0.5 | — | — | — | — |
| | Hydroxypropyl | — | — | 0.5 | — | — | — |
| | methylcellulose | | | | | | |
| | Hydroxypropyl | — | — | — | 0.5 | — | — |
| | methylcellulose stearoxy | | | | | | |
| | ether | | | | | | |
| | Polyacrylate | — | — | — | — | 0.5 | — |
| | crosspolymer-6 | | | | | | |
| | Acrylate copolymer | — | — | — | — | — | 0.5 |

### Examples 7 to 15: Preparation of Composition Containing Silicone Elastomer Powder

The silicone elastomer powder of the following Table 2 was added to the composition of Example 2 to prepare Examples 7 to 15 according to the composition ratio of Table 3. The ingredients, oil absorption capacity, and size of the added silicone elastomer powder are shown in Table 2.

The detailed preparation method is as follows.

As shown in the following Table 3, the organic UV protection agent was dissolved by heating at 80° C. and cooled to 25° C. to prepare phase A, and then oil was added to phase A to prepare phase B. Ethanol and a thickening agent were uniformly dispersed at room temperature using an AGI mixer to prepare phase C. After adding phase B to phase C, dispersion was performed at 4000 rpm for 5 minutes using a HOMO mixer to prepare phase D. After the primary dispersion of the silicone elastomer powder was performed using an AGI mixer, the secondary dispersion was performed at 3500 rpm using a HOMO mixer to prepare phase E. Thereafter, phase E was introduced into phase D and was uniformly dispersed at 4000 rpm for 3 minutes using a HOMO mixer.

TABLE 2

| | | Oil absorption | |
|---|---|---|---|
| | | capacity | Size |
| Phase | Ingredient | (ml/100 g) | (μm) |
| Powder | Dimethicone | 120-150 | 1-10 |
| | crosspolymer | | |
| | Dimethicone/vinyl | 200-250 | 1-10 |
| | dimethicone | | |
| | crosspolymer | | |
| | Vinyl | 50-70 | 1-10 |
| | dimethicone/methicone | | |
| | silsesquioxane | | |
| | crosspolymer | | |

TABLE 3

| Classification | Ingredient (wt %) | Example 7 | Example 8 | Example 9 | Example 10 | Example 11 |
|---|---|---|---|---|---|---|
| Organic UV | Ethylhexyl | 7.0 | 7.0 | 7.0 | 7.0 | 7.0 |
| protection | methoxycinnamate | | | | | |
| agent | Ethylhexyl salicylate | 3.0 | 3.0 | 3.0 | 3.0 | 3.0 |
| | Homosalate | 4.0 | 4.0 | 4.0 | 4.0 | 4.0 |
| | Octocrylene | 3.0 | 3.0 | 3.0 | 3.0 | 3.0 |
| | Diethylamino | 1.0 | 1.0 | 1.0 | 1.0 | 1.0 |
| | hydroxybenzoyl hexyl | | | | | |
| | benzoate | | | | | |
| | Butyl | 2.0 | 2.0 | 2.0 | 2.0 | 2.0 |
| | methoxydibenzoylmethane | | | | | |
| Oil | Butyl octyl salicylate | 10.0 | 8.0 | 6.0 | 10.0 | 8.0 |
| | Dicaprylyl carbonate | 6.0 | 4.0 | 2.0 | 6.0 | 4.0 |
| | Tocopheryl acetate | 1.0 | 1.0 | 1.0 | 1.0 | 1.0 |
| Ethanol | Ethanol | 45.0 | 45.0 | 45.0 | 45.0 | 45.0 |
| Thickening | Polyvinyl pyrrolidone | 3.5 | 3.5 | 3.5 | 3.5 | 3.5 |
| agent | Hydroxypropyl cellulose | 0.5 | 0.5 | 0.5 | 0.5 | 0.5 |
| Silicone | Dimethicone | 12.0 | 14.0 | 16.0 | 12.0 | 14.0 |
| elastomer | Dimethicone crosspolymer | 2.0 | 4.0 | 6.0 | — | — |
| | Dimethicone/vinyl | — | — | — | 2.0 | 4.0 |
| | dimethicone crosspolymer | | | | | |
| | Vinyl | — | — | — | — | — |
| | dimethicone/methicone | | | | | |
| | silsesquioxane | | | | | |
| | crosspolymer | | | | | |

| Classification | Example 12 | Example 13 | Example 14 | Example 15 |
|---|---|---|---|---|
| Organic UV | 7.0 | 7.0 | 7.0 | 7.0 |
| protection | | | | |
| agent | 3.0 | 3.0 | 3.0 | 3.0 |
| | 4.0 | 4.0 | 4.0 | 4.0 |
| | 3.0 | 3.0 | 3.0 | 3.0 |
| | 1.0 | 1.0 | 1.0 | 1.0 |
| | 2.0 | 2.0 | 2.0 | 2.0 |
| Oil | 6.0 | 10.0 | 8.0 | 6.0 |
| | 2.0 | 6.0 | 4.0 | 2.0 |
| | 1.0 | 1.0 | 1.0 | 1.0 |
| Ethanol | 45.0 | 45.0 | 45.0 | 45.0 |
| Thickening | 3.5 | 3.5 | 3.5 | 3.5 |
| agent | 0.5 | 0.5 | 0.5 | 0.5 |
| Silicone | 16.0 | 12.0 | 14.0 | 16.0 |
| elastomer | — | — | — | — |
| | 6.0 | — | — | — |
| | — | 2.0 | 4.0 | 6.0 |

### Experimental Example 1: Viscosity Evaluation

Table 4 shows the results of the viscosity evaluation of the compositions according to Examples 1 to 15. Viscosity measurement was performed using a Brookfield viscometer LUT under conditions of spindle No. 4 and a spin speed of 6 rpm.

TABLE 4

| | Viscosity |
|---|---|
| Classification | (cP) |
| Example 1 | 24,000 |
| Example 2 | 26,000 |
| Example 3 | 26,000 |
| Example 4 | 24,000 |
| Example 5 | 25,000 |
| Example 6 | 25,000 |
| Example 7 | 34,000 |
| Example 8 | 46,000 |
| Example 9 | 53,000 |
| Example 10 | 55,000 |
| Example 11 | 70,000 |
| Example 12 | 87,000 |
| Example 13 | 30,000 |
| Example 14 | 33,000 |
| Example 15 | 40,000 |

In Table 4, it can be seen that the viscosity was higher in the case of using mixed thickening agents (Examples 2 to 6) than in Example 1 using polyvinyl pyrrolidone alone.

Further, it can be seen that the viscosity is very high, about 30,000 cP or more in the case where the silicone elastomer powder was added (Examples 7 to 15) while the viscosity is in a range of about 24,000 to 26,000 cP in the case where the silicone elastomer powder was not added (Examples 1 to 6).

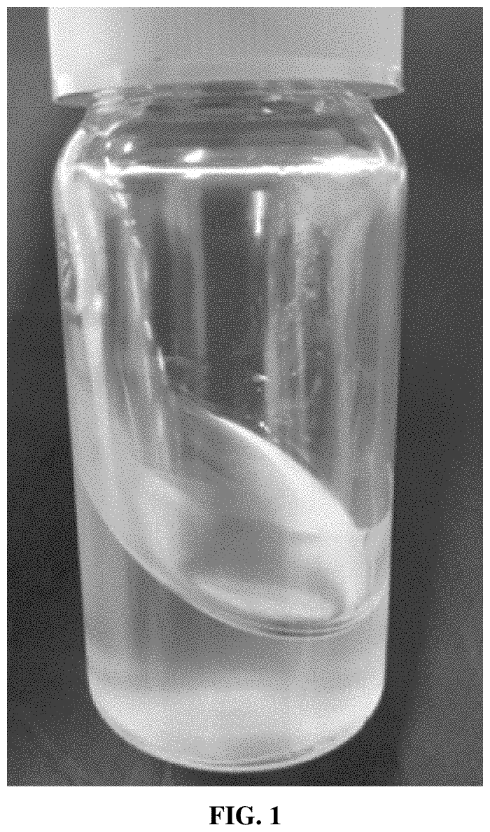

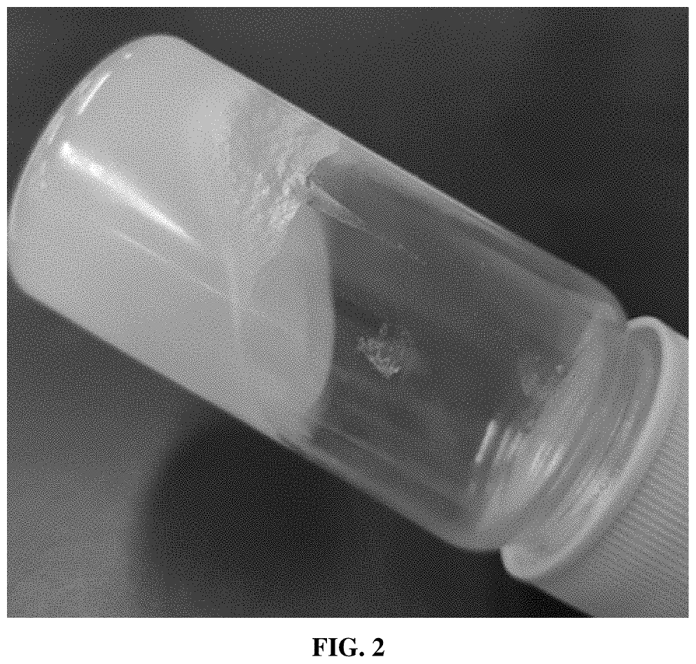

Representatively, the composition formulation of Example 2 is shown in FIG. 1, and the composition formulation of Example 8 is shown in FIG. 2. Since the viscosity of Example 8 is very high compared to Example 2, it can be seen that, even when the bottle is tilted, contents do not flow and a gel phase is stably maintained.

### Experimental Example 2: Usability Evaluation

In order to check the usability of the compositions according to Examples 1 to 15, three men and seven women in their 20s and 30s evaluated adhesion speed, glossiness, oiliness and stickiness, and the results are shown in the following Table 5.

As for the experimental method, 1 mg/cm² of each sample of the Examples were taken and applied with a width of 3 cm and a length of 2 cm to the back of measuring person's hand according to usual cosmetic habits, and the evaluation items were evaluated with 5 ratings as an evaluation scale and were scored based on a 5 point scale based on one to five points corresponding to the ratings. Usability was evaluated by calculating the average value obtained by summing the scores of ten people and excluding the deviation.

<Evaluation Items>
Adhesion speed: the higher the adhesion speed, the higher the score.
Glossiness: the lower the glossiness, the higher the score.
Oiliness: the lower the oiliness, the higher the score.
Stickiness: the lower the stickiness, the higher the freshness, the higher the score.

TABLE 5

| | Adhesion | | | |
|---|---|---|---|---|
| Classification | speed | Glossiness | Oiliness | Stickiness |
| Example 1 | 4.2 | 2.2 | 2.4 | 1.9 |
| Example 2 | 4.5 | 2.3 | 2.8 | 1.9 |
| Example 3 | 4.4 | 1.5 | 2.8 | 2.2 |
| Example 4 | 3.9 | 1.9 | 2.5 | 2.0 |
| Example 5 | 3.5 | 2.0 | 2.4 | 1.7 |
| Example 6 | 4.2 | 2.1 | 2.0 | 1.5 |
| Example 7 | 4.3 | 4.0 | 4.4 | 4.5 |
| Example 8 | 4.4 | 4.2 | 4.6 | 4.4 |
| Example 9 | 4.3 | 4.7 | 4.6 | 4.4 |
| Example 10 | 4.2 | 4.5 | 4.7 | 4.7 |
| Example 11 | 4.6 | 4.5 | 4.9 | 4.9 |
| Example 12 | 4.6 | 4.8 | 5.0 | 4.9 |
| Example 13 | 4.5 | 4.3 | 4.2 | 4.5 |
| Example 14 | 4.4 | 4.2 | 4.2 | 4.7 |
| Example 15 | 4.4 | 4.6 | 4.6 | 4.7 |

1: Very bad
2: Bad
3: Fair
4: Good
5: Very good

As can be seen from Table 5, Examples 7 to 15 including the silicone elastomer powder obtained higher scores than Examples 1 to 6 in the overall items.

Particularly, it can be seen that items of oiliness and stickiness were evaluated as poor in Examples 1 to 6, while all items were evaluated as excellent in Examples 7 to 15.

### Experimental Example 3: Absorption Power Evaluation

In order to check the absorption power for wet skin of the compositions according to Examples 1 to 15, three men and seven women in their 20s and 30s evaluated the adhesion speed and stickiness items, and the results are shown in the following Table 6.

The evaluation method was the same as the usability evaluation method. The absorption speed and the feeling of stickiness were evaluated with 5 ratings and were scored based on a 5 points scale based on one to five points corresponding to the ratings, and white cast was observed with the naked eye.

<Evaluation Items>
Absorption speed: the faster the absorption speed, the higher the score.
Stickiness: the lower the stickiness, the higher the freshness, the higher the score.

TABLE 6

| | Absorption | | White |
|---|---|---|---|
| Classification | speed | Stickiness | cast |
| Example 1 | 4.5 | 3.5 | X |
| Example 2 | 4.2 | 3.8 | X |
| Example 3 | 4.3 | 3.6 | X |
| Example 4 | 4.0 | 3.5 | X |
| Example 5 | 4.0 | 3.2 | X |
| Example 6 | 4.4 | 3.4 | X |
| Example 7 | 4.3 | 4.3 | X |
| Example 8 | 4.2 | 4.4 | X |
| Example 9 | 4.4 | 4.5 | X |
| Example 10 | 4.6 | 4.9 | X |
| Example 11 | 4.5 | 4.8 | X |
| Example 12 | 4.9 | 4.8 | X |
| Example 13 | 4.3 | 4.3 | X |
| Example 14 | 4.3 | 4.3 | X |
| Example 15 | 4.6 | 4.6 | X |

1: Very bad
2: Bad
3: Fair
4: Good
5: Very good

As a result, no white cast was observed in all Examples, but it can be seen that the absorption speed and stickiness effect in Examples 7 to 15 were superior to those of Examples 1 to 6.

Particularly, as a result of observation by the experimenter, it can be seen that when the compositions of Examples 1 to 6 were applied to wet skin, the compositions were not absorbed and thus were rubbed with fingers frequently, and the evaluation result of usability was poor due to high stickiness even after absorption. On the other hand, it can be seen that when the compositions of Examples 7 to 15 were applied to wet skin, a refreshing feeling was provided without stickiness due to the unique soft texture of the silicone elastomer powder, and thus the evaluation result of usability was very excellent.

### Experimental Example 4: Water Resistance Evaluation

Table 7 shows the results of water resistance evaluation performed on the compositions according to Examples 1 to 15.

For water resistance measurement, 1.3 mg/cm² of each sample of the Examples was taken to a PMMA plate, applied for about 30 seconds, and then dried in the dark for 15 minutes. The SPF before immersion of the dried samples was measured using a Labsphere UV 2000S In Vitro SPF analyzer. The plate was immersed for 20 minutes twice while stirring a beaker with water using a magnetic bar. After drying for 2 minutes in the dark, SPF after immersion was measured to compare the difference in SPF before and after immersion to calculate UV blocking retention.

TABLE 7

| | UV blocking |
|---|---|
| Classification | retention (%) |
| Example 1 | 23 |
| Example 2 | 28 |
| Example 3 | 21 |
| Example 4 | 20 |
| Example 5 | 29 |
| Example 6 | 21 |
| Example 7 | 66 |
| Example 8 | 65 |
| Example 9 | 69 |
| Example 10 | 70 |
| Example 11 | 75 |
| Example 12 | 77 |
| Example 13 | 69 |
| Example 14 | 71 |
| Example 15 | 74 |

As can be seen from Table 7, Examples 7 to 15 including the silicone elastomer powder have very excellent water resistance compared to Examples 1 to 6. Since the silicone elastomer powder has excellent skin adhesion properties, is hydrophobic, and is not easily wet with water, the water resistance of the composition can be improved when the silicone elastomer powder is included.

### Comparative Examples 1 to 6: Preparation of Composition Containing Other Ingredients Instead of Silicone Elastomer Powder

Comparative Examples 1 to 6 were prepared by changing the silicone elastomer ingredient of the compositions of Examples 7 to 15 to the ingredients listed in the following Table 8, which are commonly used in the related field. The preparation method is the same as in Examples 7 to 15, and the content of each ingredient is shown in Table 9.

More specifically, the ingredients listed in Table 8 are ingredients commonly used in cosmetics, where amorphous silica, silica, methyl methacrylate crosspolymer, and polymethyl silsesquioxane are in spherical powder form, dimethicone-dmethicone/vinyl dimethicone crosspolymer is a high-viscosity silicone gel, and trimethylsiloxysilicate is a silicone resin in powder form.

TABLE 8

| | | Oil | | |
|---|---|---|---|---|
| | | absorption | | |
| | | capacity | Size | Viscosity |
| Phase | Ingredient | (ml/100 g) | (μm) | (cP) |
| Powder | Amorphous silica | 350-400 | 2-5 | — |
| | Silica | 200-250 | 1-10 | — |
| | Methyl methacrylate | 100-150 | 1-10 | — |
| | crosspolymer | | | |
| | Polymethylsilsesquioxane | 50-70 | 1-10 | — |
| Gel | Dimethicone•dimethicone/ | — | — | 1.8 × 10⁵- |
| | vinyl dimethicone crosspolymer | | | 2.0 × 10⁵ |
| Powder | Trimethylsiloxysilicate | — | 1-10 | — |

TABLE 9

| | | Comparative | Comparative | Comparative | Comparative | Comparative | Comparative |
|---|---|---|---|---|---|---|---|
| Classification | Ingredient (wt %) | Example 1 | Example 2 | Example 3 | Example 4 | Example 5 | Example 6 |
| Organic UV | Ethylhexyl methoxycinnamate | 7.0 | 7.0 | 7.0 | 7.0 | 7.0 | 7.0 |
| protection | Ethylhexyl salicylate | 3.0 | 3.0 | 3.0 | 3.0 | 3.0 | 3.0 |
| agent | Homosalate | 4.0 | 4.0 | 4.0 | 4.0 | 4.0 | 4.0 |
| | Octocrylene | 3.0 | 3.0 | 3.0 | 3.0 | 3.0 | 3.0 |
| | Diethylamino hydroxybenzoyl | 1.0 | 1.0 | 1.0 | 1.0 | 1.0 | 1.0 |
| | hexyl benzoate | | | | | | |
| | Butyl methoxydibenzoylmethane | 2.0 | 2.0 | 2.0 | 2.0 | 2.0 | 2.0 |
| Oil | Butyl octyl salicylate | 9.0 | 10.0 | 10.0 | 10.0 | 10.0 | 10.0 |
| | Dicaprylyl carbonate | 5.0 | 6.0 | 6.0 | 6.0 | 6.0 | 6.0 |
| | Tocopheryl acetate | 1.0 | 1.0 | 1.0 | 1.0 | 1.0 | 1.0 |
| Ethanol | Ethanol | 45.0 | 45.0 | 45.0 | 45.0 | 45.0 | 45.0 |
| Thickening | Polyvinyl pyrrolidone | 3.5 | 3.5 | 3.5 | 3.5 | 3.5 | 3.5 |
| agent | Hydroxypropyl cellulose | 0.5 | 0.5 | 0.5 | 0.5 | 0.5 | 0.5 |
| Change in | Dimethicone | 13.0 | 10.0 | 10.0 | 10.0 | — | 10.0 |
| ingredient | Amorphous silica | 3.0 | — | — | — | — | — |
| | Silica | — | 4.0 | — | — | — | — |
| | Methyl methacrylate crosspolymer | — | — | 4.0 | — | — | — |
| | Polymethylsilsesquioxane | — | — | — | 4.0 | — | — |
| | Dimethicone(80)•dimethicone/vinyl | — | — | — | — | 14.0 | — |
| | dimethicone crosspolymer(20) | | | | | | |
| | Trimethylsiloxysilicate | — | — | — | — | — | 4.0 |

### Comparative Experimental Example 1: Formulation Stability Evaluation

The formulation stability evaluation was performed on compositions according to Examples 7 to 15 and Comparative Examples 1 to 6, and the results are shown in Table 10. Each composition was placed in a transparent plastic bottle container and maintained at a temperature of 25° C., 45° C., and CYC (−5° C. to 40° C.), and after 1 month, a change in viscosity, separation, and a change in odor according to temperature conditions were observed to evaluate formulation stability.

TABLE 10

| | Change in | | Change |
|---|---|---|---|
| Classification | viscosity | Separation | in odor |
| Example 7 | X | X | X |
| Example 8 | X | X | X |
| Example 9 | 3% Viscosity | X | X |
| | increase at | | |
| | 5° C. | | |
| Example 10 | X | X | X |
| Example 11 | X | X | X |
| Example 12 | 6% Viscosity | X | X |
| | increase at | | |
| | 5° C. | | |
| Example 13 | X | X | X |
| Example 14 | X | X | X |
| Example 15 | 3% Viscosity | X | X |
| | increase at | | |
| | 5° C. | | |
| Comparative | — | — | — |
| Example 1 | | | |
| Comparative | 40% Viscosity | Separated under | X |
| Example 2 | increase at | CYC conditions | |
| | 25° C. and | | |
| | 45° C. | | |
| Comparative | 40% Viscosity | Separated under | X |
| Example 3 | increase at | CYC conditions | |
| | 25° C. and | | |
| | 45° C. | | |
| Comparative | X | Separated under | Peculiar |
| Example 4 | | 45° C. and | odor |
| | | CYC conditions | |
| Comparative | — | — | — |
| Example 5 | | | |
| Comparative | X | Separated under | Peculiar |
| Example 6 | | all conditions | odor |

As a result, it can be seen that the compositions of Examples 7 to 15 showed a viscosity increase of less than 10%, the ingredients were not separated or had no change in odor, and a very stable formulation was maintained.

On the other hand, in the case of Comparative Example 1, the formulation was hardened due to high absorption power of the added amorphous silica, so that the formulation was not formed into a gel form.

In the case of Comparative Example 2, silica having oil absorption capacity similar to that of the silicone elastomer powder applied in Examples 10 to 12 was used, but unlike the silicone elastomer powder, viscosity increased significantly over time, and oil was re-released under varying temperature conditions, causing separation.

In Comparative Example 3, formulation separation was observed in addition to a rapid increase in viscosity. In Comparative Example 4, separation occurred, and a peculiar odor was sensed in the formulation when the formulation was stored for a long period of 3 weeks or more. In Comparative Example 5, during preparation, viscosity decreased when phases A+B+C and phase D were mixed, and the formulation was not maintained and separated eventually. It seems that the organic UV protection agent and oil having high polarity affected the swelling degree of the non-polar silicone elastomer gel, thereby completely inhibiting viscosity-forming ability. In Comparative Example 6, separation occurred during long-term storage, and a peculiar odor was sensed.

### Comparative Experimental Example 2: Usability Evaluation

The usability of Comparative Examples 1 to 6 was tested in the same manner as in Experimental Example 2, and the results are shown in the following Table 11.

TABLE 11

| | Adhesion | | | |
|---|---|---|---|---|
| Classification | speed | Glossiness | Oiliness | Stickiness |
| Comparative | — | — | — | — |
| Example 1 | | | | |
| Comparative | 3.5 | 2.5 | 2.4 | 2.3 |
| Example 2 | | | | |
| Comparative | 3.2 | 2.9 | 2.6 | 2.6 |
| Example 3 | | | | |
| Comparative | 3.4 | 2.1 | 2.7 | 1.9 |
| Example 4 | | | | |
| Comparative | — | — | — | — |
| Example 5 | | | | |
| Comparative | 3.8 | 2.2 | 2.0 | 1.1 |
| Example 6 | | | | |

1: Very bad
2: Bad
3: Fair
4: Good
5: Very good

The usability evaluation was not performed on Comparative Examples 1 and 5 because formulation was incomplete. It can be seen that stickiness was evaluated to be poor in all of Comparative Examples 2 to 4 and 6, and particularly, overall usability of Comparative Examples 6 is poor.

The UV protection cosmetic composition of the present invention has no white cast because re-emulsification does not occur even when the UV protection cosmetic composition is applied to wet skin.

In addition, viscosity is maintained above a certain level due to the mixed use of the organic thickening agent and the silicone elastomer powder, and the gel-type composition has advantages of good spreadability and easy re-application. Furthermore, the composition is not sticky or oily and can provide a fresh feeling.

Further, skin adhesion and persistence effects are high, a UV protection effect is excellent, and an excellent water resistance effect can be provided.

Although the present invention has been described in detail with reference to specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A UV protection cosmetic composition comprising an organic UV protection agent, a thickening agent, a silicone elastomer, ethanol and oil.

2. The UV protection cosmetic composition according to claim 1, wherein the silicone elastomer is in powder form.

3. The UV protection cosmetic composition according to claim 1, wherein the silicone elastomer includes one or more selected from the group consisting of dimethicone crosspolymer, dimethicone/vinyl dimethicone crosspolymer, dimethicone/vinyl dimethicone crosspolymer-silica, dimethicone/vinyl dimethicone crosspolymer-silica-dimethicone, vinyl dimethicone/methicone silsesquioxane crosspolymer and a mixture thereof.

4. The UV protection cosmetic composition according to claim 1, wherein the thickening agent includes polyvinyl pyrrolidone.

5. The UV protection cosmetic composition according to claim 4, wherein the thickening agent further includes one or more selected from the group consisting of hydroxypropyl cellulose, hydroxypropyl methylcellulose, hydroxypropyl methylcellulose stearoxy ether, acrylate/C10-30 alkyl acrylate crosspolymer, carbomer, hydroxyethyl acrylate/sodium acryloyldimethyl taurate copolymer, polyacrylate-13, polyvinyl alcohol, ammonium acryloyldimethyl taurate/VP copolymer, polyacrylate crosspolymer-6, polyacrylate crosspolymer-11, an acrylate copolymer and a mixture thereof.

6. The UV protection cosmetic composition according to claim 1, wherein the organic UV protection agent includes one or more selected from the group consisting of ethylhexyl methoxycinnamate, ethylhexyl salicylate, homosalate, octocrylene, polysilicone-15, isoamyl p-methoxycinnamate, ethylhexyl triazone, butyl methoxydibenzoylmethane, bis-ethylhexyloxyphenol methoxyphenyl triazine, diethylamino hydroxybenzoyl hexyl benzoate, 4-methylbenzylidene camphor, benzophenone-3, phenylbenzimidazole sulfonic acid, drometrizole trisiloxane, terephthalylidene dicamphor sulfonic acid, disodium phenyl dibenzimidazole tetrasulfonate and a mixture thereof.

7. The UV protection cosmetic composition according to claim 1, wherein the oil includes one or more selected from the group consisting of butyloctyl salicylate, dibutyl adipate, butylene glycol dicaprylate/dicaprate, propylene glycol dicaprylate/dicaprate, propylene glycol dicaprylate, dicaprylyl carbonate, propylheptyl caprylate, dicaprylyl ether, coco-caprylate/caprate, ethylhexyl palmitate, cyclopentasiloxane, dimethicone, disiloxane, trisiloxane, ethyl methicone, methyl trimethicone, phenyl trimethicone, caprylyl methicone, isododecane, isoeicosane, C15-19 alkanes and a mixture thereof.

8. The UV protection cosmetic composition according to claim 2, wherein the composition includes, based on a total weight of the composition, 1 to 60 wt % of the organic UV protection agent, 0.05 to 20 wt % of the thickening agent, 0.05 to 30 wt % of the silicone elastomer in powder form, 1 to 80 wt % of the ethanol and 1 to 60 wt % of the oil.

9. The UV protection cosmetic composition according to claim 2, wherein the composition includes, based on a total weight of the composition, 5 to 40 wt % of the organic UV protection agent, 0.1 to 8 wt % of the thickening agent, 0.1 to 10 wt % of the silicone elastomer in powder form, 4 to 60 wt % of the ethanol and 5 to 40 wt % of the oil.

10. The UV protection cosmetic composition according to claim 2, wherein a powder particle of the silicone elastomer has a particle diameter of 1 to 20 micrometers.

11. The UV protection cosmetic composition according to claim 1, wherein the composition is a gel formulation.

12. The UV protection cosmetic composition according to claim 1, wherein the composition has a viscosity of 24,000 cP or more.

13. The UV protection cosmetic composition according to claim 1, wherein the composition has a viscosity ranging from 30,000 to 100,000 cP.
